(19) United States
(12) Patent Application Publication
(10) Pub. No.: US 2023/0243832 A1
(43) Pub. Date: Aug. 3, 2023

(54) METHOD OF USING CIRBP AS A MOLECULAR MARKER FOR DIAGNOSIS AND TREATMENT OF PANCREATIC CANCER

(71) Applicant: Long CHEN, Kunming (CN)

(72) Inventors: Long Chen, Kunming (CN); Chengde Liao, Kunming (CN); Hongqiang Gao, Kunming (CN); Fake Yang, Kunming (CN); Yong Zha, Kunming (CN); Yongzhu Pu, Kunming (CN); Jindan Li, Kunming (CN); Chengtao Feng, Kunming (CN); Yue Wang, Kunming (CN); Rong Huang, Kunming (CN); Qiuyuan Hu, Kunming (CN)

(21) Appl. No.: 18/297,858

(22) Filed: Apr. 10, 2023

(30) Foreign Application Priority Data
Feb. 23, 2023 (CN) 202310159484.3

(51) Int. Cl. G01N 33/574; A01N 1/02

(52) U.S. Cl. CPC G01N 33/57434; A01N 1/0284; G01N 2800/7028

(57) ABSTRACT

A method of using CIRBP as a molecular marker for diagnosis and treatment of pancreatic cancer is disclosed. The method includes the following steps: S1: material preparation, S2: cell resuscitation, S3: cell culture, S4: experiment grouping, and S5: RIP to detect P CIRBP regulating TP53. For the method, the nucleoplasm expression of antibody is significantly inversely related to the tumor size of patients with pancreatic cancer, the paraneoplastic plasma expression of antibody is significantly inversely related to the diabetes history of patients with pancreatic cancer. In other words, the plasmas expression of antibody of the subgroup without a history of diabetes is significantly higher, the total survival of the pancreatic cancer patients with a high nuclear expression of the antibody is significantly better, and the survival of the pancreatic cancer with a high expression of the target gene in the database query is significantly better for inhibiting cancer.

### CROSS REFERENCE TO RELATED APPLICATION

This patent application claims the benefit and priority of Chinese Patent Application No. 202310159484.3 filed on Feb. 23, 2023, the disclosure of which is incorporated by reference herein in its entirety as part of the present application.

### TECHNICAL FIELD

The present disclosure relates to the technical field of biological detection, and more specifically, to a method of using CIRBP as a molecular marker for diagnosis and treatment of pancreatic cancer.

### BACKGROUND ART

RIP (RNA binding protein immunoprecipitation) is a technology to study the binding condition of an intracellular RNA and a protein in cells. The corresponding RNA-protein compound is precipitated by the antibody targeting the target protein, and then the RNA bound to the compound can be analyzed after separation and purification. That is, antibodies or epitope markers are adopted to capture endogenous RNA-binding proteins in the nucleus or cytoplasm to prevent the binding of non-specific RNA. The RNA binding protein and the bound RNA thereof are separated together by immunoprecipitation. The binding RNA sequence is identified by the method of microarray (RIP-chip), quantitative RT-PCR or high-throughput sequencing (RIP-Seq). The target protein is a stress response protein, which can migrate from a nucleus to a cytoplasm during the stress response. In addition, the target protein can also be secreted from the cell and function as a molecular model related to damage outside the cell, promote an inflammatory damage, and play an important role in acute and chronic inflammation.

### SUMMARY

A method of using CIRBP as a molecular marker for diagnosis and treatment of pancreatic cancer is provided, which includes the following steps:

S1: material preparation, preparation of reagent cell culture reagents: fetal calf serum, DMEM-high glucose medium, RPMI-1640 medium, penicillin-streptomycin, PBS potassium phosphate buffer, Trypsin-EDTA (0.25%) phenol red and experimental consumable 6 well plate cell culture plates, 12 well plate cell culture plates, 24 well plate cell culture plates, 48 well plate cell culture plates, 96 well plate cell culture plates, and Pasteur pipettes;

S2: cell resuscitation; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cryopreservation tube for rapidly thawing, quickly putting the cryopreservation tube into the pre-heated water bath kettle for rapid thawing, and constantly shaking to rapidly melt a liquid in the cryopreservation tube, and taking out the cryopreservation tube when there is still a little unmelted liquid in the cryopreservation tube, wiping an outer wall of the cryopreservation tube with an alcohol cotton ball, bringing the cryopreservation tube into the ultra-clean worktable for preparation of a cell suspension, transferring cells into a 15 mL centrifuge tube, adding a preheated culture medium drop by drop, shaking the centrifuge tube at the same time; performing centrifugation after an amount of the added culture medium is more than 10 mL, performing centrifugation on a low-speed centrifuge at 800 rpm for 5 minutes; pipetting a supernatant, and then resuspending the cells with 1 mL of culture medium to obtain a cell suspension, separating the cell suspension into a culture dish, and putting the culture dish into an incubator containing CO₂ at 37° C. for culture, wherein the time of changing the solution depends on a cell sedimentation rate;

S3: cell culture; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cell culture bottle with sterile operation, opening the bottle lid, pipetting the original culture solution off, washing the cells with PBS one to two times, adding a trypsin-EDTA solution (1 mL/25 cm², 2 mL/75 cm²), gently washing the bottom of the cell dish, pipetting the trypsin-EDTA solution off, placing in a 37° C. incubator for 2-3 minutes, and tapping the wall of the culture bottle to make most of the cells fall off, observing under an inverted microscope, and when the cells are to be separated and present a round granulate, adding an appropriate amount of fresh culture medium containing serum to terminate the trypsin effect;

S4: experiment grouping, wherein the detection items are RIP to detect CIRBP regulating TP53, PCR to detect CIRBP, TP53, wb, CIRBP, ACSL4, PTGS2, NOX1, GPX4, FTH1, Prussian blue staining, laser confocal detection for mitochondrial reactive oxygen ROS, glutathione GSH detection, flow apoptosis detection, cell proliferation growth curve detection;

S5: RIP to detect P CIRBP regulating TP53.

Preferably, a flow of the experiment in the step S4 is: a: cell lysate acquisition; b: preparation of magnetic beads; c: chromatin shearing; d: RNA binding protein immunoprecipitation; e: RNA purification.

Preferably, in the step S4, the procedure of R PCR to detect CIRBP and TP53 is: sample total RNA extraction, three-step fluorescent quantitative RT-PCR reaction, fluorescent quantitative PCR reaction, and fluorescent quantitative RT-PCR data analysis.

Preferably, the procedure of sample total RNA extraction is: taking an appropriate amount of sample material and grinding same with liquid nitrogen, then adding 1000 uL Trizol for full lysis, and then transferring same to a 2 mL centrifuge tube, shaking the 2 mL centrifuge tube for 2 min, then performing incubation at 15-30° C. for 5 min, adding 200 uL chloroform (0.2 mL chloroform per 1 mL Trizol), closing the tube lid, thoroughly shaking 15-60 s, not more than 12000 g, performing centrifugation at 2-8° C. for 15 min, and carefully pipetting the supernatant to another centrifuge tube, never pipetting to a middle layer, and adding 500 uL isopropanol (0.5 mL isopropanol per 1 mL Trizol).

Preferably, in the step S4, a procedure of wb detecting CIRBP, ACSL4, PTGS2, NOX1, GPX4 and FTH1 is: a: collecting a protein sample, formulating an SDS-PAGE gel, and performing sample processing; adding an appropriate amount of concentrated SDS-PAGE protein loading buffer solution to the collected protein sample, performing loading and electrophoresis, and after cooling to room temperature, directly loading the protein sample into an SDS-PAGE gel loading hole, and heating same at 100° C. or a boiling water bath for 3-5 minutes to fully denature the protein; b: transfer, wherein generally, if a standard wet transfer apparatus of Bio-Rad is adopted, the transfer current can be set to be 300-400 mA, and a transfer duration is 30-60 minutes; alternatively, the transfer can be performed overnight at 15-20 mA; the specific transfer time depends on the size of the target protein; the larger the molecular weight of the target protein is, the longer the required transfer time is, and the smaller the molecular weight of the target protein is, the shorter the required transfer time is; c: blocking, after the transfer, immediately placing the protein membrane in a pre-prepared Western washing solution, performing rinse for 1-2 minutes to wash off a transfer solution on the membrane, shaking slowly on a shaker, and carrying out blocking at room temperature for 60 minutes, wherein for some antibodies with high background, the blocking can be performed overnight at 4° C.; d: incubation of the primary antibody; aspirating off the blocking solution with a miniature desktop vacuum pump or a dropper or the like, and immediately adding the diluted primary antibody, incubating same for one hour at room temperature or 4° C. with slow shaking on a side-swing shaker; if the effect of incubation of the primary antibody for one hour is not good, performing incubation with slow shaking at 4° C., recovering the primary antibody, adding Western wash solution, and washing same with slow shaking on the side-swing shaker for 5-10 minutes, aspirating off the washing solution, adding the washing solution for washing for 5-10 minutes, wherein washing is performed for 3 times in total, and if the background of the result is relatively high, the washing duration can be prolonged properly and the number of washing times can be increased; e: dilution of secondary antibody labeled with horseradish peroxidase (HRP) with Western secondary antibody diluent in appropriate ratios, the secondary antibody being selected on the basis of the secondary antibody, aspirating off the blocking solution with a miniature desktop vacuum pump or a dropper or the like, and immediately adding the diluted secondary antibody, incubating same for one hour at room temperature or 4° C. with slow shaking on a side-swing shaker, recovering the secondary antibody, adding Western wash solution, and washing same with slow shaking on the side-swing shaker for 5-10 minutes, aspirating the washing solution, adding the washing solution for washing for 5-10 minutes, wherein washing is performed for 3 times in total, and if the background of the result is relatively high, the washing duration can be prolonged properly and the number of washing times can be increased; f: detection of proteins; adopting ECL reagents such as BeyoECL Plus to detecting proteins, wherein exposure may be performed by using a dedicated X-ray exposure cartridge, and film development may be performed by an automatic X-ray film developer; g: gel image analysis; scanning or photographing the film, and analyzing a molecular weight and a net optical density value of a target band using a gel image processing system.

Preferably, in the step S4, the procedure of the Prussian blue staining is: a: performing fixation with 95% alcohol for not less than 60 minutes; b. washing with distilled water; c: taking and mixing 2% aqueous solution of potassium hydroferrocyanide and 2% aqueous solution of hydrochloric acid in equal parts, dropping same on a smear for 10-20 minutes; d: washing with distilled water; e: counterstaining with nuclear fast red; f: washing with water, dehydrating, clearing, and mounting.

Preferably, in the step S4, the procedure of the laser confocal detection for mitochondrial reactive oxygen ROS is: a: formulation of MitoTracker™ red storage solution; b: formulation of MitoTracker &trade red working solution; c: fluorescent labelling of mitochondria; d: loading ROS probe; e: Mitosox loading into cells; f: washing; g: microscopy.

Preferably, in the step S4, the procedure of the cell proliferation growth curve detection is: a: taking cells from each treatment group for the following experiments; b: digesting the cells, spreading the cells, counting same, adjusting a cell concentration to 1×10⁵ cells/mL, dividing the cells into the 96 well plate at 100 uL per well, that is, 1×10⁴ cells per well; c: collecting adherent cells at each time point for detection after adhering; d: collecting cells at various time points (0 h, 24 h, 48 h, 72 h) and adding CCK-8 solution (Beyotime, Cat. No. 00037) at a ratio of 1/10, that is, 10 ul of detection solution to 100 uL of incubation solution; e: after incubation for 4 h, reading the plate by a microplate reader and reading OD450 data by CCK-8 detection.

The present disclosure provides a method of using CIRBP as a molecular marker for diagnosis and treatment of pancreatic cancer. The method has the following beneficial effects.

For the method of using CIRBP as a molecular marker for diagnosis and treatment of pancreatic cancer, the plasmas expression of antibody is significantly lower in pancreatic cancer than in paracancerous tissues, the nucleoplasm expression of antibody is significantly inversely related to the tumor size of patients with pancreatic cancer, the paraneoplastic plasma expression of antibody is significantly inversely related to the diabetes history of patients with pancreatic cancer. In other words, the plasmas expression of antibody of the subgroup without a history of diabetes is significantly higher, the total survival of the pancreatic cancer patients with a high nuclear expression of the antibody is significantly better, and the survival of the pancreatic cancer with a high expression of the target gene in the database query is significantly better for inhibiting cancer.

### BRIEF DESCRIPTION OF THE DRAWINGS

FIG. 1 is a schematic diagram of the experimental principle of the present disclosure;

FIG. 2 is a schematic diagram of primer design for detecting binding positions of the present disclosure;

FIG. 3 is a schematic diagram of original data of detection and its analyzing of the present disclosure;

FIG. 4 is a schematic diagram of the amount of template for RIP CIRBP amplification in three samples of the present disclosure;

FIG. 5 is a schematic diagram of RIP CIRBP amplification electrophoresis of the present disclosure;

FIG. 6 is a schematic diagram of a CIRBP amplification curve of the present disclosure;

FIG. 7 is a schematic diagram of a CIRBP dissolution curve of the present disclosure;

FIG. 8 is a schematic diagram of a TP53 amplification curve of the present disclosure;

FIG. 9 is a schematic diagram of a TP53 dissolution curve of the present disclosure;

FIG. 10 is a schematic diagram of raw data and its calculation of the present disclosure;

FIG. 11 is a schematic diagram of the cell low-temperature sensitive proliferation rate of the present disclosure.

### DETAILED DESCRIPTION OF THE EMBODIMENTS

Technical solutions of the present disclosure will be clearly and completely described below with reference to the embodiments. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

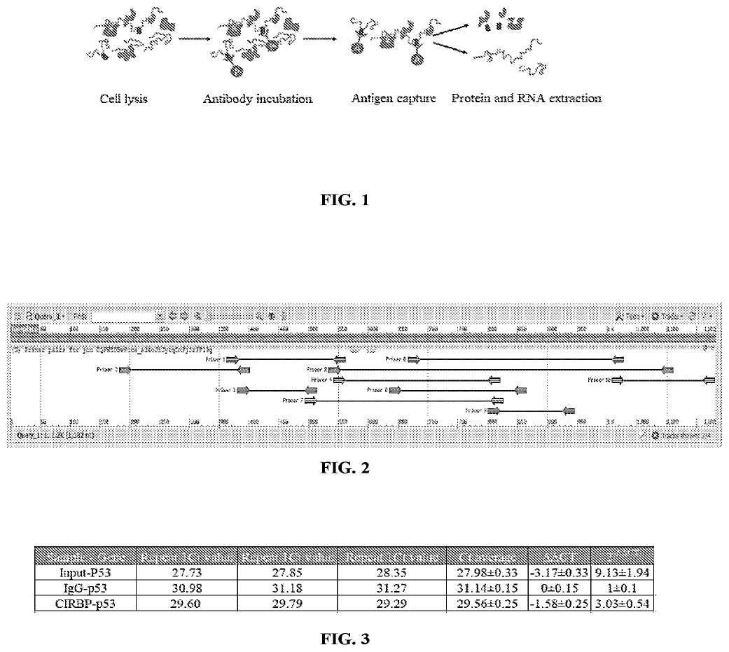

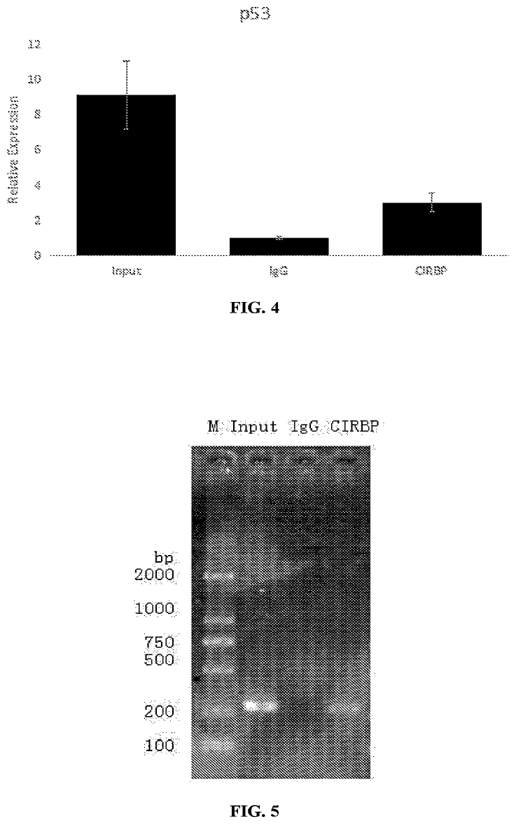

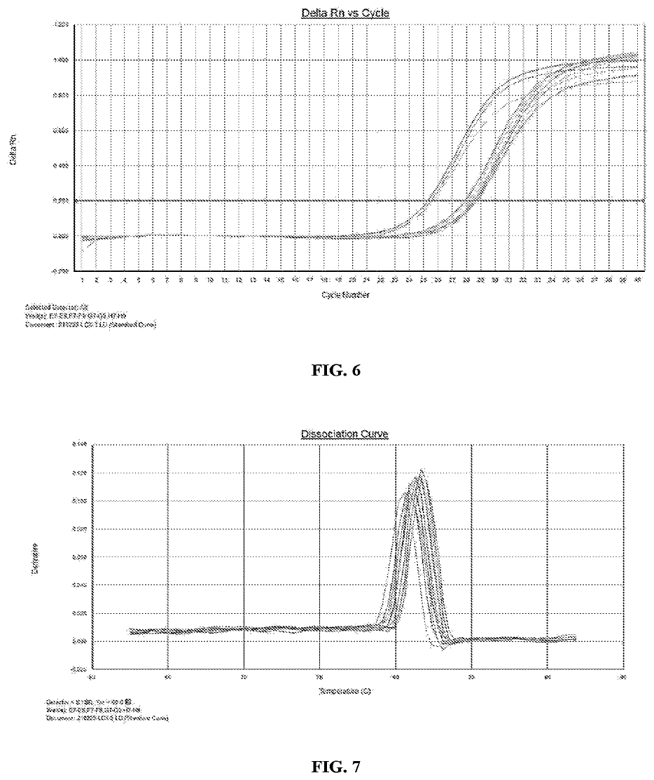

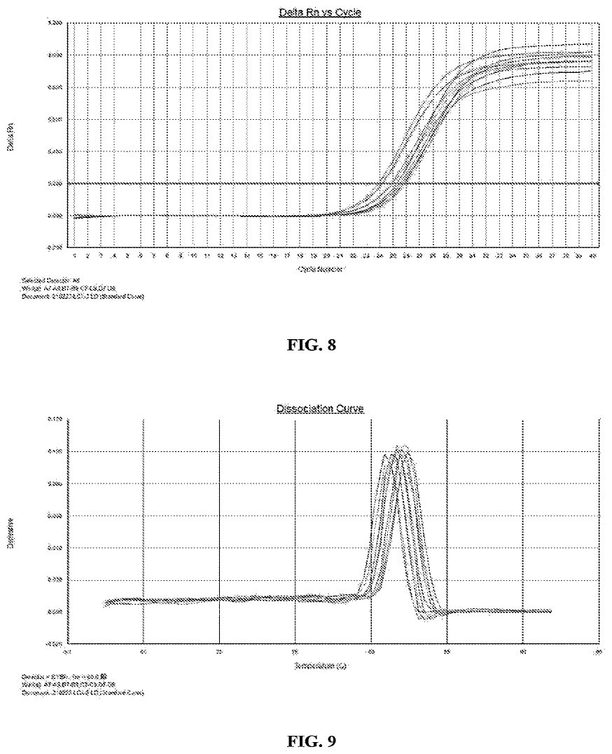

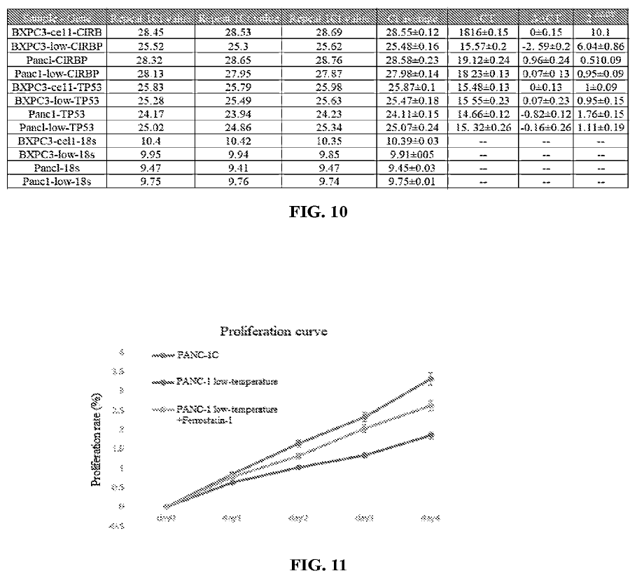

Referring to FIG. 1- FIG. 11, the embodiment of the present disclosure provides a technical solution: a method of using CIRBP as a molecular marker for diagnosis and treatment of pancreatic cancer. The method includes the steps as follows:

S1: material preparation, preparation of reagent cell culture reagents: fetal calf serum, DMEM-high glucose medium, RPMI-1640 medium, penicillin-streptomycin, PBS potassium phosphate buffer, Trypsin-EDTA (0.25%) phenol red and experimental consumable 6 well plate cell culture plates, 12 well plate cell culture plates, 24 well plate cell culture plates, 48 well plate cell culture plates, 96 well plate cell culture plates, and Pasteur pipettes;

S2: cell resuscitation; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cryopreservation tube for rapidly thawing, quickly putting the cryopreservation tube into the pre-heated water bath kettle for rapid thawing, and constantly shaking to rapidly melt a liquid in the cryopreservation tube, and taking out the cryopreservation tube when there is still a little unmelted liquid in the cryopreservation tube, wiping an outer wall of the cryopreservation tube with an alcohol cotton ball, bringing the cryopreservation tube into the ultra-clean worktable for preparation of a cell suspension, transferring cells into a 15 mL centrifuge tube, adding a preheated culture medium drop by drop, shaking the centrifuge tube at the same time; performing centrifugation after an amount of the added culture medium is more than 10 mL, performing centrifugation on a low-speed centrifuge at 800 rpm for 5 minutes; pipetting a supernatant, and then resuspending the cells with 1 mL of culture medium to obtain a cell suspension, separating the cell suspension into a culture dish, and putting the culture dish into an incubator containing CO₂ at 37° C. for culture, wherein the time of changing the solution depends on a cell sedimentation rate;

S3: cell culture; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cell culture bottle with sterile operation, opening the bottle lid, pipetting the original culture solution off, washing the cells with PBS one to two times, adding a trypsin-EDTA solution (1 mL/25 cm², 2 mL/75 cm²), gently washing the bottom of the cell dish, pipetting the trypsin-EDTA solution off, placing in a 37° C. incubator for 2-3 minutes, and tapping the wall of the culture bottle to make most of the cells fall off, observing under an inverted microscope, and when the cells are to be separated and present a round granulate, adding an appropriate amount of fresh culture medium containing serum to terminate the trypsin effect;

S4: experiment grouping, wherein the detection items are RIP to detect CIRBP regulating TP53, PCR to detect CIRBP, TP53, wb, CIRBP, ACSL4, PTGS2, NOX1, GPX4, FTH1, Prussian blue staining, laser confocal detection for mitochondrial reactive oxygen ROS, glutathione GSH detection, flow apoptosis detection, cell proliferation growth curve detection;

S5: RIP to detect P CIRBP regulating TP53.

The flow of the experiment in the step S4 is: a: cell lysate acquisition; b: preparation of magnetic beads; c: chromatin shearing; d: RNA binding protein immunoprecipitation; e: RNA purification.

In the step S4, the procedure of R PCR to detect CIRBP and TP53 is: sample total RNA extraction, three-step fluorescent quantitative RT-PCR reaction, fluorescent quantitative PCR reaction, and fluorescent quantitative RT-PCR data analysis.

The procedure of sample total RNA extraction is: taking an appropriate amount of sample material and grinding same with liquid nitrogen, then adding 1000 uL Trizol for full lysis, and then transferring same to a 2 mL centrifuge tube, shaking the 2 mL centrifuge tube for 2 min, then performing incubation at 15-30° C. for 5 min, adding 200 uL chloroform (0.2 mL chloroform per 1 mL Trizol), closing the tube lid, thoroughly shaking 15-60 s, not more than 12000 g, performing centrifugation at 2-8° C. for 15 min, and carefully pipetting the supernatant to another centrifuge tube, never pipetting to a middle layer, and adding 500 uL isopropanol (0.5 mL isopropanol per 1 mL Trizol).

In the step S4, the procedure of wb detecting CIRBP, ACSL4, PTGS2, NOX1, GPX4 and FTH1 is: a: collecting a protein sample, formulating an SDS-PAGE gel, and performing sample processing; adding an appropriate amount of concentrated SDS-PAGE protein loading buffer solution to the collected protein sample, performing loading and electrophoresis, and after cooling to room temperature, directly loading the protein sample into an SDS-PAGE gel loading hole, and heating same at 100° C. or a boiling water bath for 3-5 minutes to fully denature the protein; b: transfer, wherein generally, if a standard wet transfer apparatus of Bio-Rad is adopted, the transfer current can be set to be 300-400 mA, and a transfer duration is 30-60 minutes; alternatively, the transfer can be performed overnight at 15-20 mA; the specific transfer time depends on the size of the target protein; the larger the molecular weight of the target protein is, the longer the required transfer time is, and the smaller the molecular weight of the target protein is, the shorter the required transfer time is; c: blocking, after the transfer, immediately placing the protein membrane in a pre-prepared Western washing solution, performing rinse for 1-2 minutes to wash off a transfer solution on the membrane, shaking slowly on a shaker, and carrying out blocking at room temperature for 60 minutes, wherein for some antibodies with high background, the blocking can be performed overnight at 4° C.; d: incubation of the primary antibody; aspirating off the blocking solution with a miniature desktop vacuum pump or a dropper or the like, and immediately adding the diluted primary antibody, incubating same for one hour at room temperature or 4° C. with slow shaking on a side-swing shaker; if the effect of incubation of the primary antibody for one hour is not good, performing incubation with slow shaking at 4° C., recovering the primary antibody, adding Western wash solution, and washing same with slow shaking on the side-swing shaker for 5-10 minutes, aspirating off the washing solution, adding the washing solution for washing for 5-10 minutes, wherein washing is performed for 3 times in total, and if the background of the result is relatively high, the washing duration can be prolonged properly and the number of washing times can be increased; e: dilution of secondary antibody labeled with horseradish peroxidase (HRP) with Western secondary antibody diluent in appropriate ratios, the secondary antibody being selected on the basis of the secondary antibody, aspirating off the blocking solution with a miniature desktop vacuum pump or a dropper or the like, and immediately adding the diluted secondary antibody, incubating same for one hour at room temperature or 4° C. with slow shaking on a side-swing shaker, recovering the secondary antibody, adding Western wash solution, and washing same with slow shaking on the side-swing shaker for 5-10 minutes, aspirating the washing solution, adding the washing solution for washing for 5-10 minutes, wherein washing is performed for 3 times in total, and if the background of the result is relatively high, the washing duration can be prolonged properly and the number of washing times can be increased; f: detection of proteins; adopting ECL reagents such as BeyoECL Plus to detecting proteins, wherein exposure may be performed by using a dedicated X-ray exposure cartridge, and film development may be performed by an automatic X-ray film developer; g: gel image analysis; scanning or photographing the film, and analyzing a molecular weight and a net optical density value of a target band using a gel image processing system.

In the step S4, the procedure of the Prussian blue staining is: a: performing fixation with 95% alcohol for not less than 60 minutes; b. washing with distilled water; c: taking and mixing 2% aqueous solution of potassium hydroferrocyanide and 2% aqueous solution of hydrochloric acid in equal parts, dropping same on a smear for 10-20 minutes; d: washing with distilled water; e: counterstaining with nuclear fast red; f: washing with water, dehydrating, clearing, and mounting.

In the step S4, the procedure of the laser confocal detection for mitochondrial reactive oxygen ROS is: a: formulation of MitoTracker™ red storage solution; b: formulation of MitoTracker &trade red working solution; c: fluorescent labelling of mitochondria; d: loading ROS probe; e: Mitosox loading into cells; f: washing; g: microscopy.

In the step S4, the procedure of the cell proliferation growth curve detection is: a: taking cells from each treatment group for the following experiments; b: digesting the cells, spreading the cells, counting same, adjusting a cell concentration to 1×10⁵ cells/mL, dividing the cells into the 96 well plate at 100 uL per well, that is, 1×10⁴ cells per well; c: collecting adherent cells at each time point for detection after adhering; d: collecting cells at various time points (0 h, 24 h, 48 h, 72 h) and adding CCK-8 solution (Beyotime, Cat. No. 00037) at a ratio of 1/10, that is, 10 ul of detection solution to 100 uL of incubation solution; e: after incubation for 4 h, reading the plate by a microplate reader and reading OD450 data by CCK-8 detection.

While the basic principles, main features and advantages of the present disclosure have been shown and described, it is apparent to those skilled in the art that the present disclosure is not limited to the details of the foregoing exemplary embodiments, but can be embodied in other specific forms without departing from the spirit or essential features of the present disclosure. Therefore, from whatever point on, the embodiments should be viewed as exemplary, and also non limiting, and the scope of the present disclosure is defined by the appended claims rather than the foregoing illustration. All changes that come within the meaning and range of equivalency of the claims are therefore intended to be embraced therein. Any reference mark in the claims shall not be regarded as limiting the claims involved.

In addition, it should be understood that although the description is given according to the embodiments, each embodiment does not only include an independent technical solution. This narrative manner of the specification is only for clarity. Those skilled in the art should make the specification as a whole, and the technical solutions in each embodiment may also be appropriately combined to form other embodiments that can be understood by those skilled in the art.

What is claimed is:

1. A method of using CIRBP as a molecular marker for diagnosis and treatment of pancreatic cancer, comprising following steps:
S1. material preparation, preparation of reagent cell culture reagents: fetal calf serum, DMEM-high glucose medium, RPMI-1640 medium, penicillin-streptomycin, PBS potassium phosphate buffer, Trypsin-EDTA (0.25%) phenol red and experimental consumable 6 well plate cell culture plates, 12 well plate cell culture plates, 24 well plate cell culture plates, 48 well plate cell culture plates, 96 well plate cell culture plates, and Pasteur pipettes;
S2. cell resuscitation; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cryopreservation tube for rapidly thawing, quickly putting the cryopreservation tube into the pre-heated water bath kettle for rapid thawing, and constantly shaking to rapidly melt a liquid in the cryopreservation tube, and taking out the cryopreservation tube when there is still a little unmelted liquid in the cryopreservation tube, wiping an outer wall of the cryopreservation tube with an alcohol cotton ball, bringing the cryopreservation tube into the ultra-clean worktable for preparation of a cell suspension, transferring cells into a 15 mL centrifuge tube, adding a preheated culture medium drop by drop, shaking the centrifuge tube at the same time; performing centrifugation after an amount of the added culture medium is more than 10 mL, performing centrifugation on a low-speed centrifuge at 800 rpm for 5 minutes; pipetting a supernatant, and then resuspending the cells with 1 mL of culture medium to obtain a cell suspension, separating the cell suspension into a culture dish, and putting the culture dish into an incubator containing CO₂ at 37° C. for culture, wherein the time of changing the solution depends on a cell sedimentation rate;
S3. cell culture; pre-heating a water bath kettle to 37° C., and using 75% alcohol to wipe an ultra-clean worktable surface after ultraviolet radiation for 30 min, placing sterilized centrifuge tubes, suction tubes, culture bottles, etc. in sequence in the ultra-clean worktable, taking out a cell culture bottle with sterile operation, opening the bottle lid, pipetting the original culture solution off, washing the cells with PBS one to two times, adding a trypsin-EDTA solution (1 mL/25 cm², 2 mL/75 cm²), gently washing the bottom of the cell dish, pipetting the trypsin-EDTA solution off, placing in a 37° C. incubator for 2-3 minutes, and tapping the wall of the culture bottle to make most of the cells fall off, observing under an inverted microscope, and when the cells are to be separated and present a round granulate, adding an appropriate amount of fresh culture medium containing serum to terminate the trypsin effect;
S4. experiment grouping, wherein the detection items are RIP to detect CIRBP regulating TP53, PCR to detect CIRBP, TP53, wb, CIRBP, ACSL4, PTGS2, NOX1, GPX4, FTH1, Prussian blue staining, laser confocal detection for mitochondrial reactive oxygen ROS, glutathione GSH detection, flow apoptosis detection, cell proliferation growth curve detection;
S5. RIP to detect P CIRBP regulating TP53.

2. The method of claim 1, wherein a flow of the experiment In the step S4 is:
a: cell lysate acquisition;
b: preparation of magnetic beads;
c: chromatin shearing;
d: RNA binding protein immunoprecipitation;
e: RNA purification.

3. The method of claim 1, wherein In the step S4, a procedure of R PCR to detect CIRBP and TP53 comprises: sample total RNA extraction, three-step fluorescent quantitative RT-PCR reaction, fluorescent quantitative PCR reaction, and fluorescent quantitative RT-PCR data analysis.

4. The method of claim 3, wherein a procedure of sample total RNA extraction comprises:
taking an appropriate amount of sample material and grinding same with liquid nitrogen, then adding 1000 uL Trizol for full lysis, and then transferring same to a 2 mL centrifuge tube, shaking the 2 mL centrifuge tube for 2 min, then performing incubation at 15-30° C. for 5 min, adding 200 uL chloroform (0.2 mL chloroform per 1 mL Trizol), closing the tube lid, thoroughly shaking 15-60 s, not more than 12000 g, performing centrifugation at 2-8° C. for 15 min, and carefully pipetting the supernatant to another centrifuge tube, never pipetting to a middle layer, and adding 500 uL isopropanol (0.5 mL isopropanol per 1 mL Trizol).

5. The method of claim 1, wherein In the step S4, a procedure of wb detecting CIRBP, ACSL4, PTGS2, NOX1, GPX4 and FTH1 comprises:
a: collecting a protein sample, formulating an SDS-PAGE gel, and performing sample processing; adding an appropriate amount of concentrated SDS-PAGE protein loading buffer solution to the collected protein sample, performing loading and electrophoresis, and after cooling to room temperature, directly loading the protein sample into an SDS-PAGE gel loading hole, and heating same at 100° C. or a boiling water bath for 3-5 minutes to fully denature the protein;
b: transfer, wherein generally, if a standard wet transfer apparatus of Bio-Rad is adopted, the transfer current can be set to be 300-400 mA, and a transfer duration is 30-60 minutes; alternatively, the transfer can be performed overnight at 15-20 mA; the specific transfer time depends on the size of the target protein; the larger the molecular weight of the target protein is, the longer the required transfer time is, and the smaller the molecular weight of the target protein is, the shorter the required transfer time is;
c: blocking, after the transfer, immediately placing the protein membrane in a pre-prepared Western washing solution, performing rinse for 1-2 minutes to wash off a transfer solution on the membrane, shaking slowly on a shaker, and carrying out blocking at room temperature for 60 minutes, wherein for some antibodies with high background, the blocking can be performed overnight at 4° C.;
d: incubation of the primary antibody; aspirating off the blocking solution with a miniature desktop vacuum pump or a dropper or the like, and immediately adding the diluted primary antibody, incubating same for one hour at room temperature or 4° C. with slow shaking on a side-swing shaker; if the effect of incubation of the primary antibody for one hour is not good, performing incubation with slow shaking at 4° C., recovering the primary antibody, adding Western wash solution, and washing same with slow shaking on the side-swing shaker for 5-10 minutes, aspirating off the washing solution, adding the washing solution for washing for 5-10 minutes, wherein washing is performed for 3 times in total, and if the background of the result is relatively high, the washing duration can be prolonged properly and the number of washing times can be increased;
e: dilution of secondary antibody labeled with horseradish peroxidase (HRP) with Western secondary antibody diluent in appropriate ratios, the secondary antibody being selected on the basis of the secondary antibody, aspirating off the blocking solution with a miniature desktop vacuum pump or a dropper or the like, and immediately adding the diluted secondary antibody, incubating same for one hour at room temperature or 4° C. with slow shaking on a side-swing shaker, recovering the secondary antibody, adding Western wash solution, and washing same with slow shaking on the side-swing shaker for 5-10 minutes, aspirating the washing solution, adding the washing solution for washing for 5-10 minutes, wherein washing is performed for 3 times in total, and if the background of the result is relatively high, the washing duration can be prolonged properly and the number of washing times can be increased;
f: detection of proteins; adopting ECL reagents such as BeyoECL Plus to detecting proteins, wherein exposure may be performed by using a dedicated X-ray exposure cartridge, and film development may be performed by an automatic X-ray film developer;
g: gel image analysis; scanning or photographing the film, and analyzing a molecular weight and a net optical density value of a target band using a gel image processing system.

6. The method of claim 1, wherein in the step S4, a procedure of the Prussian blue staining comprises: a: performing fixation with 95% alcohol for not less than 60 minutes; b. washing with distilled water; c: taking and mixing 2% aqueous solution of potassium hydroferrocyanide and 2% aqueous solution of hydrochloric acid in equal parts, dropping same on a smear for 10-20 minutes; d: washing with distilled water; e: counterstaining with nuclear fast red; f: washing with water, dehydrating, clearing, and mounting.

7. The method of claim 1, wherein in the step S4, a procedure of the laser confocal detection for mitochondrial reactive oxygen ROS comprises: a: formulation of MitoTracker™ red storage solution; b: formulation of MitoTracker &trade red working solution; c: fluorescent labelling of mitochondria; d: loading ROS probe; e: Mitosox loading into cells; f: washing; g: microscopy.

8. The method of claim 1, wherein in the step S4, a procedure of the cell proliferation growth curve detection comprises: a: taking cells from each treatment group for the following experiments; b: digesting the cells, spreading the cells, counting same, adjusting a cell concentration to 1×10⁵ cells/mL, dividing the cells into the 96 well plate at 100 uL per well, that is, 1×10⁴ cells per well; c: collecting adherent cells at each time point for detection after adhering; d: collecting cells at various time points (0 h, 24 h, 48 h, 72 h) and adding CCK-8 solution (Beyotime, Cat. No. 00037) at a ratio of 1/10, that is, 10 ul of detection solution to 100 uL of incubation solution; e: after incubation for 4 h, reading the plate by a microplate reader and reading OD450 data by CCK-8 detection.
